(19) United States
(12) Patent Application Publication
(10) Pub. No.: US 2005/0131546 A1
(43) Pub. Date: Jun. 16, 2005

(54) Replacement ligamentum teres femoris

(76) Inventor: Amit Mor, Rehovot (IL)

Correspondence Address: DEKEL PATENT LTD., DAVID KLEIN, BEIT HAROF'IM, 18 MENUHA VENAHALA STREET, ROOM 27, REHOVOT, 76209, IL

(21) Appl. No.: 10/734,123

(22) Filed: Dec. 15, 2003

(51) Int. Cl. A61F 2/32

(52) U.S. Cl. 623/22.11

(57) ABSTRACT

A hip joint prosthesis including a replacement ligamentum teres femoris. The replacement ligamentum teres femoris may be attached to a replacement (or natural) femoral head and/or to a replacement (or natural) acetabular structure.

### FIELD OF THE INVENTION

The present invention relates generally to an enarthrodial prosthesis, and particularly to a replacement ligamentum teres femoris for a hip joint prosthesis.

### BACKGROUND OF THE INVENTION

The hip joint (whose scientific name is the coxal articulation) is an enarthrosis (ball-and-socket joint). Many kinds of hip joint prostheses are known. For a better understanding of a hip joint prosthesis, a basic description of the human hip joint is presented here with reference to FIGS. 1-3.

The femoral head (head of the thigh bone) articulates with the acetabulum of the innominate bone, known in non-technical terms as the socket of the hipbone. The innominate bone in the area of the hip joint is made of three portions: the upper portion is called the ilium, the middle portion is called the pubis and the lower portion is called the ischium. The femoral head is connected to the innominate bone by a plurality of ligaments. The ligaments shown in FIG. 1 are the ilio-femoral ligaments and the pubo-femoral ligament. There is also an ischio-femoral ligament on the posterior part of the joint, not seen in the figures. The femoral head articulates with a fibrous rim of the acetabulum called the cotyloid ligament or glenoidal labrum. A capsular ligament (seen dissected in FIG. 2) surrounds the neck of the femur and is attached to the margin of the acetabulum posteriorly beyond the cotyloid ligament and anteriorly to the outer margin of the cotyloid ligament.

The femoral head is connected to the acetabular notch by a ligament called the ligamentum teres femoris or ligamentum teres for short. Quoting from Gray's Anatomy: “The ligamentum teres femoris is a triangular, somewhat flattened band implanted by its apex into the antero-superior part of the fovea capitis femoris; its base is attached by two bands, one into either side of the acetabular notch, and between these bony attachments it blends with the transverse ligament. It is ensheathed by the synovial membrane, and varies greatly in strength in different subjects; occasionally only the synovial fold exists, and in rare cases even this is absent. The ligament is made tense when the thigh is semiflexed and the limb then adducted or rotated outward; it is, on the other hand, relaxed when the limb is abducted. It has, however, but little influence as a ligament.”

The statement that the ligamentum teres has “little influence as a ligament” is accepted in the medical literature to the present. Quoting from a medical dictionary website (www.books.md/L/dic/ligamentumteresfemoris. php) that defines ligamentum teres as follows: “A flattened ligament that passes from the fovea in the head of the femur to the borders of the acetabular notch (transverse acetabular ligament); developmentally, an artery passes to the head of the femur with the ligament which may or may not persist into adulthood; the ligament does not contribute to the integrity of the joint or control movements there.”

Generally a hip joint replacement of the prior art involves replacing the natural femoral head with a metallic or plastic artificial femoral head, which is fixedly attached to a stem. The stem is generally inserted in the femur and the femoral head articulates with the acetabulum, if still intact, or some other depression, artificial or natural, in the innominate bone. Some or all of the ilio-femoral, pubo-femoral and ischio-femoral ligaments may be removed to provide access to the femoral head and acetabulum. The ligamentum teres is generally severed and removed. No known hip joint prostheses have or are connected to a ligamentum teres, perhaps because of the prevalent belief that “the ligament does not contribute to the integrity of the joint or control movements there”.

### SUMMARY OF THE INVENTION

The present invention seeks to provide a novel hip joint prosthesis, which includes a replacement ligamentum teres femoris, as is described more in detail hereinbelow.

The following is provided as background information for better appreciation of the present invention, but the invention is not limited to the validity of this information.

Proprioception refers to the ability to know where a body part is located in space and to recognize movements of body parts (such as fingers and toes, feet and hands, legs and arms). Kinesthesia is a related term, and refers to the sensation by which position, weight, muscle tension and movement are perceived. In some of the medical literature, proprioception refers to the conscious and unconscious appreciation of joint position, while kinesthesia refers to the sensation of joint velocity and acceleration. Proprioception is often used interchangeably with kinesthesia, and herein as well, the terms will be used interchangeably.

The neuromuscular control system of the body integrates peripheral sensations relative to joint loads and processes these signals into coordinated motor responses. This muscle activity serves to protect joint structures from excessive strain.

Certain mechanoreceptors are present throughout the soft tissues of the musculoskeletal system, which interact with the central nervous system and coordinate body movements, postural alignment, and balance. Mechanoreceptors are located in the muscles, tendons, ligaments, joint capsules and the skin. These nerve fibers provide information to the brain regarding the status and function of the musculoskeletal system. The mechanoreceptors send electrical signals along peripheral nerves to the spinal cord. The electrical signals travel via the spinal cord to the brain where the signals are interpreted to recognize movements of body parts, muscle tension, movement and the like.

Some examples of mechanoreceptors for controlling the muscular system include muscle spindles. Muscle spindles are found interspersed within the contractile fibers of skeletal muscles, with the highest concentration in the central portion of each muscle. Muscle spindle fibers respond to changes in the length of muscles. These nerve endings provide the central nervous system information used to maintain muscle tone and the correct muscle tension on opposite sides of each joint.

Fibrous tissues that surround and protect most joints generally contain a variety of sensory nerve endings for proprioception and kinesthesia. The input from these sensory nerve endings provides the central nervous system information regarding the location, stretch, compression, tension, acceleration, and rotation of the joint.

The present inventors postulate that contrary to the abovementioned statements of the medical literature, the ligamentum teres does indeed play a role, perhaps an important role, in the function of the hip joint, particularly biomechanical and in proprioception and/or kinesthesia. However, it is emphasized that the invention is not limited in any way to the correctness or incorrectness of this postulation.

There is thus provided in accordance with an embodiment of the present invention a hip joint prosthesis including a replacement ligamentum teres femoris. The replacement ligamentum teres femoris may be attached to a replacement (or natural) femoral head and/or to a replacement (or natural) acetabular notch. The replacement ligamentum teres femoris may be attached to any part of the femur (e.g., head, neck, stem, body) and to any part of the acetabulum (not just the notch) and/or pelvis, including natural and artificial femurs, acetabula and pelvises.

In accordance with an embodiment of the present invention the replacement ligamentum teres femoris may include one attachment zone for attachment to a femoral head and at least one attachment zone to acetabular structure.

Further in accordance with an embodiment of the present invention the replacement ligamentum teres femoris may extend outwards from a portion of the replacement femoral head corresponding to a fovea (or other portion) of a natural femoral head.

Still further in accordance with an embodiment of the present invention the replacement femoral head extends from a stem insertable into a femur. The stem may include an alignment device adapted to orient the replacement femoral head in a predefined orientation.

### BRIEF DESCRIPTION OF THE DRAWINGS

The present invention will be understood and appreciated more fully from the following detailed description taken in conjunction with the drawing in which:

FIG. 1 is a simplified illustration of a right-side hip joint, showing the outer ligaments;

FIG. 2 is a simplified illustration of the right-side hip joint, showing the ligamentum teres and the acetabulum;

FIG. 3 is a simplified illustration of the left-side hip joint, showing the ligamentum teres by cutting away from the floor of the acetabulum;

FIG. 4 is a simplified illustration of a hip joint prosthesis, constructed and operative in accordance with an embodiment of the present invention; and

FIG. 5 is a simplified illustration of a hip joint prosthesis, constructed and operative in accordance with another embodiment of the present invention.

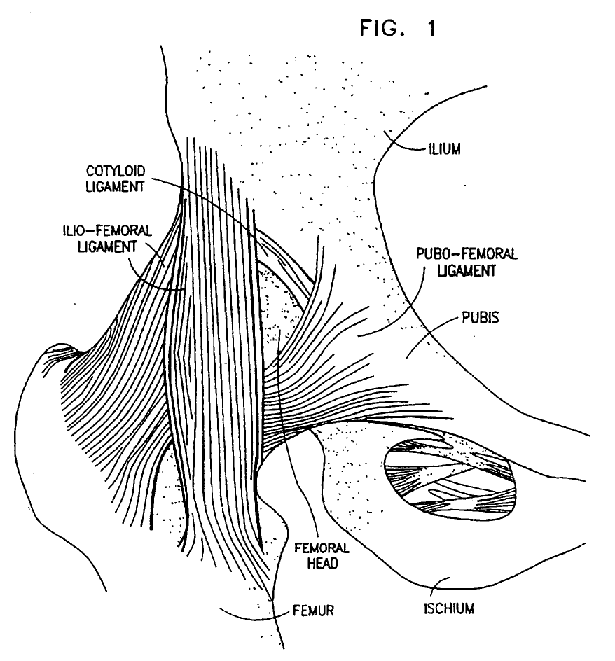

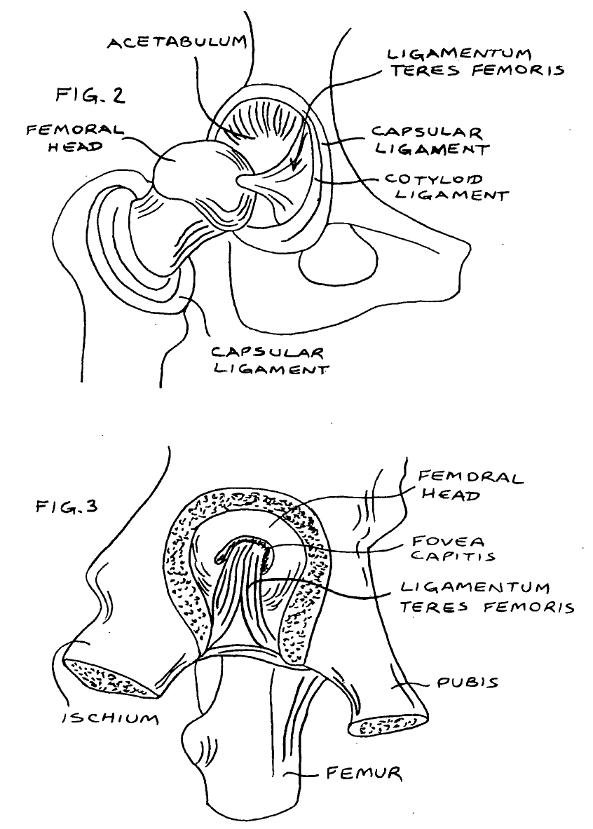

### DETAILED DESCRIPTION OF EMBODIMENTS

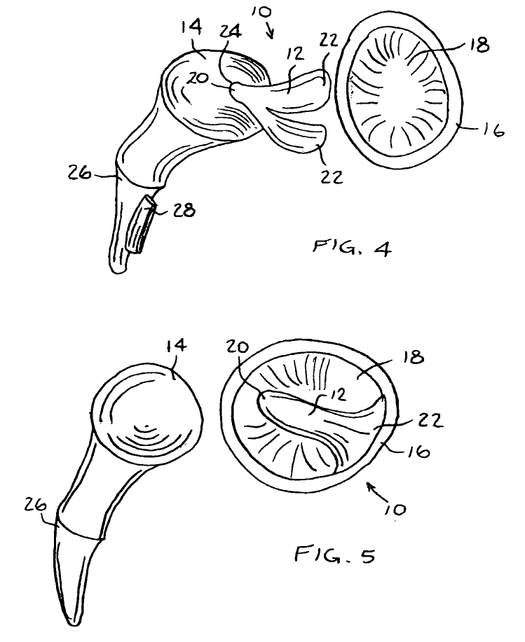

Reference is now made to FIG. 4, which illustrates a hip joint prosthesis 10, constructed and operative in accordance with an embodiment of the present invention.

The prosthesis 10 may comprise a replacement ligamentum teres femoris 12, which may be attached to a replacement femoral head 14. Alternatively, replacement ligamentum teres femoris 12 may be attached to a natural femoral head. The replacement ligamentum teres femoris 12 may be stretchable and may be constructed of any suitable medically-safe elastomer, such as but not limited to, polyurethane, polyurethane bionate, silicone and the like, or may be obtained or made from a natural ligament or other natural tissue or substance (e.g., some other ligamentum teres femoris and the like). The properties of the elastomer may be selected or engineered to mimic the properties of a natural ligamentum teres femoris.

The replacement ligamentum teres femoris 12 may be of any shape or size, such as but not limited to, a wire, cord, string, ligament, band, ribbon, and the like.

The replacement ligamentum teres femoris 12 may be attached to acetabular structure, such as but not limited to, a replacement acetabular notch 16. Notch 16 may be associated with an artificial or replacement acetabulum 18. Alternatively, the replacement ligamentum teres femoris 12 may be attached to a natural acetabular notch. Alternatively, the replacement ligamentum teres femoris 12 may be attached to the replacement acetabulum 18 or some structure of the innominate bone or neighboring tissue or bones (e.g., attached to any portion of the pelvis, directly or through an orifice). It is noted that the replacement ligamentum teres femoris 12 of the present invention does not have to be attached to the same place on the femoral head and acetabular structure as the natural ligamentum teres femoris. Any attachment zone is within the scope of the invention.

Attachment of the replacement ligamentum teres femoris 12 to either or both of the femoral head 14 and the acetabular structure may be effected by any convenient method, such as but not limited to, mechanical fasteners, bonding with adhesive, or suturing with stitches, or an EndoButton CL Fixation Device (commercially available from Smith & Nephew, Inc. Andover, Mass.) for example.

The replacement ligamentum teres femoris 12 may comprise one attachment zone 20 for attachment to the femoral head 14 and one or more attachment zones 22 (two such attachments zones 22 are illustrated in FIG. 4; one is illustrated in FIG. 5) to the acetabular structure. The replacement ligamentum teres femoris 12 may extend outwards from a portion 24 of the replacement femoral head 14 corresponding to a fovea of a natural femoral head.

It is noted that FIG. 4 illustrates a femoral head prosthesis with the replacement ligamentum teres femoris 12 attached to the femoral head 14 and ready for attachment to the replacement acetabulum 18. In contrast, FIG. 5 illustrates an acetabular prosthesis with the replacement ligamentum teres femoris 12 attached to the replacement acetabular notch 16 and ready for attachment to the replacement femoral head 14.

The replacement femoral head 14 may extend from a stem 26 insertable into a femur (not shown). Stem 26 may include an alignment device 28 that can orient the replacement femoral head 14 in a predefined orientation. For example, alignment device 28 may comprise a key or lug that protrudes outwards from stem 26 which may fit in a corresponding notch formed in the femur (not shown). One purpose of alignment device 28 would be to align the replacement ligamentum teres femoris 12 jutting from the replacement femoral head 14 to point in the correct direction of its attachment to the acetabular notch 16.

It will be appreciated by persons skilled in the art that the present invention is not limited by what has been particularly shown and described hereinabove. Rather the scope of the present invention includes both combinations and subcombinations of the features described hereinabove as well as modifications and variations thereof which would occur to a person of skill in the art upon reading the foregoing description and which are not in the prior art.

1. A hip joint prosthesis comprising:
a replacement ligamentum teres femoris non-slidingly attached to a replacement femoral head.

2. (canceled)

3. The prosthesis according to claim 1, further comprising a replacement acetabular structure to which said replacement ligamentum teres femoris is attached.

4. The prosthesis according to claim 1, wherein said replacement ligamentum teres femoris comprises one attachment zone for attachment to a femoral head and at least one attachment zone to acetabular structure.

5. The prosthesis according to claim 1, wherein said replacement ligamentum teres femoris extends outwards from a portion of said replacement femoral head corresponding to a fovea of a natural femoral head.

6. The prosthesis according to claim 1, wherein said replacement femoral head extends from a stem insertable into a femur.

7. The prosthesis according to claim 6, wherein said stem comprises an alignment device adapted to orient said replacement femoral head in a predefined orientation.

8. The prosthesis according to claim 1, wherein said replacement ligamentum teres femoris is stretchable.

9. The prosthesis according to claim 1, wherein said replacement ligamentum teres femoris does not pass through said replacement femoral head.
